SEQUENCE LISTING (124 sequences)

SEQ ID NO: 1 - RNA, 21 nt, Homo sapiens
acuggacuuggagucagaagg

SEQ ID NO: 2 - RNA, 66 nt, Homo sapiens
agggcuccugacuccagguccuguguguuaccuagaaauagcacuggacuuggagucagaaggccu

SEQ ID NO: 3 - RNA, 21 nt, Homo sapiens
aggggugcuaucugugauuga

SEQ ID NO: 4 - RNA, 99 nt, Homo sapiens
gaaacugggcucaaggugaggggugcuaucugugauugagggacaugguuaauggaauugucucacacagaaaucgcacccgucaccuuggccuacuua

SEQ ID NO: 5 - RNA, 21 nt, Homo sapiens
cgcgggugcuuacugacccuu

SEQ ID NO: 6 - RNA, 121 nt, Homo sapiens
cacuccuacccgggucggaguuagcucaagcgguuaccuccucaugccggacuuucuaucuguccaucucugugcugggguucgagacccgcgggugcuuacugacccuuuuaugcaauaa

SEQ ID NO: 7 - RNA, 21 nt, Homo sapiens
ucagugcaugacagaacuugg

SEQ ID NO: 8 - RNA, 87 nt, Homo sapiens
ugucccccccggcccagguucugugauacacuccgacucgggcucuggagcagucagugcaugacagaacuugggcccggaaggacc

SEQ ID NO: 9 - RNA, 22 nt, Homo sapiens
cagugcaaugaugaaagggcau

SEQ ID NO: 10 - RNA, 82 nt, Homo sapiens
ggccugcccgacacucuuucccuguugcacuacuauaggccgcugggaagcagugcaaugaugaaagggcaucggucagguc

SEQ ID NO: 11 - RNA, 21 nt, Homo sapiens
uaccacaggguagaaccacgg

SEQ ID NO: 12 - RNA, 100 nt, Homo sapiens
ugugucucucucuguguccugccagugguuuuacccuaugguagguuacgucaugcuguucuaccacaggguagaaccacggacaggauaccggggcacc

SEQ ID NO: 13 - RNA, 21 nt, Homo sapiens
cuagacugaagcuccuugagg

SEQ ID NO: 14 - RNA, 90 nt, Homo sapiens
uuuccugcccucgaggagcucacagucuaguaugucucauccccuacuagacugaagcuccuugaggacagggauggucauacucaccuc

SEQ ID NO: 15 - RNA, 22 nt, Homo sapiens
aaaagcuggguugagagggcga

SEQ ID NO: 16 - RNA, 82 nt, Homo sapiens
gcuucgcuccccuccgccuucucuucccgguucuucccggagucgggaaaagcuggguugagagggcgaaaaaggaugaggu

SEQ ID NO: 17 - RNA, 21 nt, Homo sapiens
caacaccagucgaugggcugu

SEQ ID NO: 18 - RNA, 72 nt, Homo sapiens
ugucggguagcuuaucagacugauguugacuguugaaucucauggcaacaccagucgaugggcugucugaca

SEQ ID NO: 19 - RNA, 22 nt, Homo sapiens
ugaccgauuucuccugguguuc

SEQ ID NO: 20 - RNA, 88 nt, Homo sapiens
aucucuuacacaggcugaccgauuucuccugguguucagagucuguuuuugucuagcaccauuugaaaucgguuaugauguaggggga

SEQ ID NO: 21 - RNA, 22 nt, Homo sapiens
aaaccguuaccauuacugaguu

SEQ ID NO: 22 - RNA, 72 nt, Homo sapiens
cuugggaauggcaaggaaaccguuaccauuacugaguuuaguaaugguaaugguucucuugcuauacccaga

SEQ ID NO: 23 - RNA, 22 nt, Homo sapiens
uagcuuaucagacugauguuga

SEQ ID NO: 24 - RNA, 72 nt, Homo sapiens; identical to SEQ ID NO: 18

SEQ ID NO: 25 - RNA, 21 nt, Homo sapiens
caagucacuagugguuccguu

SEQ ID NO: 26 - RNA, 81 nt, Homo sapiens
gggcuuucaagucacuagugguuccguuuaguagaugauugugcauuguuucaaaauggugcccuagugacuacaaagccc

SEQ ID NO: 27 - RNA, 22 nt, Homo sapiens
uggcagugucuuagcugguugu

SEQ ID NO: 28 - RNA, 110 nt, Homo sapiens
ggccagcugugaguguuucuuuggcagugucuuagcugguuguugugagcaauaguaaggaagcaaucagcaaguauacugcccuagaagugcugcacguuguggggccc

SEQ ID NO: 29 - RNA, 22 nt, Homo sapiens
uagcaccauuugaaaucgguua

SEQ ID NO: 30 - RNA, 21 nt, Homo sapiens
uucaaguaauucaggauaggu

SEQ ID NO: 31 - RNA, 22 nt, Homo sapiens
ugagguaguaguuuguacaguu

SEQ ID NO: 32 - RNA, 21 nt, Homo sapiens
uaaagugcugacagugcagau

SEQ ID NO: 33 - RNA, 21 nt, Homo sapiens
uacaguacugugauaacugaa

SEQ ID NO: 34 - RNA, 22 nt, Homo sapiens
uguaaacauccuugacuggaag

SEQ ID NO: 35 - RNA, 23 nt, Homo sapiens
uagcaccauuugaaaucaguguu

SEQ ID NO: 36 - RNA, 22 nt, Homo sapiens
agggacgggacgcggugcagug

SEQ ID NO: 37 - RNA, 23 nt, Homo sapiens
cgcauccccuagggcauuggugu

SEQ ID NO: 38 - RNA, 23 nt, Homo sapiens
aacauucauugcugucggugggu

SEQ ID NO: 39 - RNA, 22 nt, Homo sapiens
cagcagcaauucauguuuugaa

SEQ ID NO: 40 - RNA, 22 nt, Homo sapiens
cauugcacuugucucggucuga

SEQ ID NO: 41 - RNA, 23 nt, Homo sapiens
ucuggcuccgugucuucacuccc

SEQ ID NO: 42 - RNA, 22 nt, Homo sapiens
uauugcacuugucccggccugu

SEQ ID NO: 43 - RNA, 22 nt, Homo sapiens
aacuggcccucaaagucccgcu

SEQ ID NO: 44 - RNA, 22 nt, Homo sapiens
uauugcacucgucccggccucc

SEQ ID NO: 45 - RNA, 22 nt, Homo sapiens
uaauacugccugguaaugauga

SEQ ID NO: 46 - RNA, 22 nt, Homo sapiens
uaacacugucugguaacgaugu

SEQ ID NO: 47 - RNA, 22 nt, Homo sapiens
ucagugcacuacagaacuuugu

SEQ ID NO: 48 - RNA, 23 nt, Homo sapiens
agcugguguugugaaucaggccg

SEQ ID NO: 49 - RNA, 21 nt, Homo sapiens
uucacaguggcuaaguuccgc

SEQ ID NO: 50 - RNA, 21 nt, Homo sapiens
aucacauugccagggauuucc

SEQ ID NO: 51 - RNA, 23 nt, Homo sapiens
uaaggugcaucuagugcagauag

SEQ ID NO: 52 - RNA, 77 nt, Homo sapiens
ccgggacccaguucaaguaauucaggauagguugugugcuguccagccuguucuccauuacuuggcucggggaccgg

SEQ ID NO: 53 - RNA, 84 nt, Homo sapiens
aggcugagguaguaguuuguacaguuugagggucuaugauaccacccgguacaggagauaacuguacaggccacugccuugcca

SEQ ID NO: 54 - RNA, 82 nt, Homo sapiens
ccugccggggcuaaagugcugacagugcagauagugguccucuccgugcuaccgcacuguggguacuugcugcuccagcagg

SEQ ID NO: 55 - RNA, 75 nt, Homo sapiens
ugcccuggcucaguuaucacagugcugaugcugucuauucuaaagguacaguacugugauaacugaaggauggca

SEQ ID NO: 56 - RNA, 79 nt, Homo sapiens
acuguccuuuuucgguuaucaugguaccgaugcuguauaucugaaagguacaguacugugauaacugaagaaugguggu

SEQ ID NO: 57 - RNA, 92 nt, Homo sapiens
gggcagucuuugcuacuguaaacauccuugacuggaagcuguaagguguucagaggagcuuucagucggauguuuacagcggcaggcugcca

SEQ ID NO: 58 - RNA, 81 nt, Homo sapiens
cuucaggaagcugguuucauauggugguuuagauuuaaauagugauugucuagcaccauuugaaaucaguguucuuggggg

SEQ ID NO: 59 - RNA, 81 nt, Homo sapiens
cuucuggaagcugguuucacaugguggcuuagauuuuuccaucuuuguaucuagcaccauuugaaaucaguguuuuaggag

SEQ ID NO: 60 - RNA, 96 nt, Homo sapiens
cgggccccgggcgggcgggagggacgggacgcggugcaguguuguuuuuucccccgccaauauugcacucgucccggccuccggcccccccggccc

SEQ ID NO: 61 - RNA, 83 nt, Homo sapiens
cugacuaugccuccccgcauccccuagggcauugguguaaagcuggagacccacugccccaggugcugcuggggguuguaguc

SEQ ID NO: 62 - RNA, 110 nt, Homo sapiens
ccugugcagagauuauuuuuuaaaaggucacaaucaacauucauugcugucgguggguugaacuguguggacaagcucacugaacaaugaaugcaacuguggccccgcuu

SEQ ID NO: 63 - RNA, 89 nt, Homo sapiens
cugauggcugcacucaacauucauugcugucgguggguuugagucugaaucaacucacugaucaaugaaugcaaacugcggaccaaaca

SEQ ID NO: 64 - RNA, 98 nt, Homo sapiens
cgaggggauacagcagcaauucauguuuugaaguguucuaaaugguucaaaacgugaggcgcugcuauacccccucguggggaagguagaaggugggg

SEQ ID NO: 65 - RNA, 84 nt, Homo sapiens
ggccaguguugagaggcggagacuugggcaauugcuggacgcugcccugggcauugcacuugucucggucugacagugccggcc

SEQ ID NO: 66 - RNA, 89 nt, Homo sapiens
gccggcgcccgagcucuggcuccgugucuucacucccgugcuuguccgaggagggagggagggacgggggcugugcuggggcagcugga

SEQ ID NO: 67 - RNA, 78 nt, Homo sapiens
cuuucuacacagguugggaucgguugcaaugcuguguuucuguaugguauugcacuugucccggccuguugaguuugg

SEQ ID NO: 68 - RNA, 75 nt, Homo sapiens
ucaucccuggguggggauuuguugcauuacuuguguucuauauaaaguauugcacuugucccggccuguggaaga

SEQ ID NO: 69 - RNA, 83 nt, Homo sapiens
guggucucagaaucgggguuuugagggcgagaugaguuuauguuuuauccaacuggcccucaaagucccgcuuuuggggucau

SEQ ID NO: 70 - RNA, 95 nt, Homo sapiens
ccagcucgggcagccguggccaucuuacugggcagcauuggauggagucaggucucuaauacugccugguaaugaugacggcggagcccugcacg

SEQ ID NO: 71 - RNA, 90 nt, Homo sapiens
ccgggccccugugagcaucuuaccggacagugcuggauuucccagcuugacucuaacacugucugguaacgauguucaaaggugacccgc

SEQ ID NO: 72 - RNA, 68 nt, Homo sapiens
gaggcaaaguucugagacacuccgacucugaguaugauagaagucagugcacuacagaacuuugucuc

SEQ ID NO: 73 - RNA, 99 nt, Homo sapiens
cccuggcauggugugguggggcagcugguguugugaaucaggccguugccaaucagagaacggcuacuucacaacaccagggccacaccacacuacagg

SEQ ID NO: 74 - RNA, 84 nt, Homo sapiens
cguugcugcagcugguguugugaaucaggccgacgagcagcgcauccucuuacccggcuauuucacgacaccaggguugcauca

SEQ ID NO: 75 - RNA, 78 nt, Homo sapiens
cugaggagcagggcuuagcugcuugugagcaggguccacaccaagucguguucacaguggcuaaguuccgccccccag

SEQ ID NO: 76 - RNA, 73 nt, Homo sapiens
ggccggcugggguuccuggggaugggauuugcuuccugucacaaaucacauugccagggauuuccaaccgacc

SEQ ID NO: 77 - RNA, 71 nt, Homo sapiens
uguucuaaggugcaucuagugcagauagugaaguagauuagcaucuacugcccuaagugcuccuucuggca

SEQ ID NO: 78 - DNA, 28 bp, artificial sequence
  Synthetic
cagtcatttggctagcaccatttgaaat

SEQ ID NO: 79 - DNA, 28 bp, artificial sequence
  Synthetic
cagtcatttggcttcaagtaattcagga

SEQ ID NO: 80 - DNA, 28 bp, artificial sequence
  Synthetic
cagtcatttgggtgaggtagtagtttgt

SEQ ID NO: 81 - DNA, 28 bp, artificial sequence
  Synthetic
cagtcatttggctaaagtgctgacagtg

SEQ ID NO: 82 - DNA, 28 bp, artificial sequence
  Synthetic
cagtcatttggctacagtactgtgataa

SEQ ID NO: 83 - DNA, 28 bp, artificial sequence
  Synthetic
cagtcatttggctgtaaacatccttgac

SEQ ID NO: 84 - DNA, 28 bp, artificial sequence
  Synthetic
cagtcatttggctagcaccatttgaaat

SEQ ID NO: 85 - DNA, 28 bp, artificial sequence
  Synthetic
cagtcatttgggtaatactgcctggtaa

SEQ ID NO: 86 - DNA, 28 bp, artificial sequence
  Synthetic
cagtcatttgggtaacactgtctggtaa

SEQ ID NO: 87 - DNA, 28 bp, artificial sequence
  Synthetic
cagtcatttggctcagtgcactacagaa

SEQ ID NO: 88 - DNA, 22 bp, artificial sequence
  Synthetic
agggacgggacgcggtgcagtg

SEQ ID NO: 89 - DNA, 28 bp, artificial sequence
  Synthetic
cagtcatttggccgcatcccctagggca

SEQ ID NO: 90 - DNA, 28 bp, artificial sequence
  Synthetic
cagtcatttgggaacattcattgctgtc

SEQ ID NO: 91 - DNA, 28 bp, artificial sequence
  Synthetic
cagtcatttggccagcagcaattcatgt

SEQ ID NO: 92 - DNA, 28 bp, artificial sequence
  Synthetic
cagtcatttggccattgcacttgtctcg

SEQ ID NO: 93 - DNA, 28 bp, artificial sequence
  Synthetic
cagtcatttggctctggctccgtgtctt

SEQ ID NO: 94 - DNA, 28 bp, artificial sequence
  Synthetic
cagtcatttggctattgcacttgtcccg

SEQ ID NO: 95 - DNA, 28 bp, artificial sequence
  Synthetic
cagtcatttggcaactggccctcaaagt

SEQ ID NO: 96 - DNA, 28 bp, artificial sequence
  Synthetic
cagtcatttgggtattgcactcgtcccg

SEQ ID NO: 97 - DNA, 28 bp, artificial sequence
  Synthetic
cagtcatttggcagctggtgttgtgaat

SEQ ID NO: 98 - DNA, 28 bp, artificial sequence
  Synthetic
cagtcatttggcttcacagtggctaagt

SEQ ID NO: 99 - DNA, 28 bp, artificial sequence
  Synthetic
cagtcatttggcatcacattgccaggga

SEQ ID NO: 100 - DNA, 28 bp, artificial sequence
  Synthetic
cagtcatttggctaaggtgcatctagtg

SEQ ID NO: 101 - DNA, 23 bp, artificial sequence
  Synthetic
ccgttttttttttttaaccgatt

SEQ ID NO: 102 - DNA, 23 bp, artificial sequence
  Synthetic
ccgttttttttttttacctatcc

SEQ ID NO: 103 - DNA, 23 bp, artificial sequence
  Synthetic
ccgttttttttttttactgtaca

SEQ ID NO: 104 - DNA, 23 bp, artificial sequence
  Synthetic
ccgttttttttttttatctgcac

SEQ ID NO: 105 - DNA, 23 bp, artificial sequence
  Synthetic
ccgttttttttttttcagttatc

SEQ ID NO: 106 - DNA, 23 bp, artificial sequence
  Synthetic
ccgttttttttttttcttccagt

SEQ ID NO: 107 - DNA, 24 bp, artificial sequence
  Synthetic
tccgttttttttttttaacactga

SEQ ID NO: 108 - DNA, 23 bp, artificial sequence
  Synthetic
ccgttttttttttttcatcatta

SEQ ID NO: 109 - DNA, 23 bp, artificial sequence
  Synthetic
ccgttttttttttttaacatcgt

SEQ ID NO: 110 - DNA, 23 bp, artificial sequence
  Synthetic
ccgttttttttttttacaaagtt

SEQ ID NO: 111 - DNA, 18 bp, artificial sequence
  Synthetic
aaaaccgatagtgagtcg

SEQ ID NO: 112 - DNA, 23 bp, artificial sequence
  Synthetic
ccgttttttttttttacaccaat

SEQ ID NO: 113 - DNA, 23 bp, artificial sequence
  Synthetic
ccgttttttttttttacccaccg

SEQ ID NO: 114 - DNA, 23 bp, artificial sequence
  Synthetic
ccgttttttttttttcaaaacat

SEQ ID NO: 115 - DNA, 23 bp, artificial sequence
  Synthetic
ccgttttttttttttcagaccga

SEQ ID NO: 116 - DNA, 23 bp, artificial sequence
  Synthetic
ccgttttttttttttgggagtga

SEQ ID NO: 117 - DNA, 22 bp, artificial sequence
  Synthetic
cgttttttttttttacaggccg

SEQ ID NO: 118 - DNA, 22 bp, artificial sequence
  Synthetic
cgttttttttttttagcgggac

SEQ ID NO: 119 - DNA, 22 bp, artificial sequence
  Synthetic
cgttttttttttttggaggccg

SEQ ID NO: 120 - DNA, 22 bp, artificial sequence
  Synthetic
cgttttttttttttcggcctga

SEQ ID NO: 121 - DNA, 23 bp, artificial sequence
  Synthetic
ccgttttttttttttgcggaact

SEQ ID NO: 122 - DNA, 23 bp, artificial sequence
  Synthetic
ccgttttttttttttggaaatcc

SEQ ID NO: 123 - DNA, 23 bp, artificial sequence
  Synthetic
ccgttttttttttttctatctgc

SEQ ID NO: 124 - DNA, 22 bp, artificial sequence
  Synthetic
gcgagcacagaattaatacgac

(19) United States
(12) Patent Application Publication
(10) Pub. No.: US 2013/0123138 A1
(43) Pub. Date: May 16, 2013

(54) COMPOSITIONS AND METHODS FOR PROGNOSIS OF MESOTHELIOMA

(71) Applicants: Harvey Pass, Bronxville, NY (US); Moshe Hoshen, Jerusalem (IL); Yaron Goren, Kefar Hass (IL)

(72) Inventors: Harvey Pass, Bronxville, NY (US); Moshe Hoshen, Jerusalem (IL); Yaron Goren, Kefar Hass (IL)

(73) Assignee: NEW YORK UNIVERSITY

(21) Appl. No.: 13/811,550

(22) PCT Filed: Jun. 9, 2011
(86) PCT No.: PCT/IL11/00452

§ 371 Date: Jan. 22, 2013

Related U.S. Application Data
(60) Provisional application No. 61/367,419, filed on Jul. 25, 2010.
(60) Provisional application No. 61/417,905, filed on Nov. 30, 2010.

(51) Int. Cl. C12Q 1/68

(52) U.S. Cl. CPC C12Q 1/6886
USPC 506/9; 435/6.12; 435/6.11

(57) ABSTRACT

Disclosed are compositions and methods for the prognosis of mesothelioma patients after surgical operation. Specifically the disclosure provides microRNA (miRNA) molecules associated with prognosis of mesothelioma, as well as various nucleic acid molecules relating thereto or derived therefrom. The disclosure further provides altered expression of miRNAs that are associated with good or poor prognosis of mesothelioma.

### CROSS REFERENCE TO RELATED APPLICATIONS

The present application claims priority under 35 U.S.C. §119(e) to U.S. Provisional Applications No. 61/367,419 filed Jul. 25, 2010 and No. 61/417,905 filed Nov. 30, 2010, which are herein incorporated by reference in their entirety.

### FIELD OF THE INVENTION

The invention relates to compositions and methods for the prognosis of mesothelioma patients after surgical operation. Specifically the invention relates to microRNA molecules associated with the prognosis of mesothelioma, as well as various nucleic acid molecules relating thereto or derived therefrom.

### BACKGROUND OF THE INVENTION

In recent years, microRNAs (miRs, miRNAs) have emerged as an important novel class of regulatory RNA, which has profound impact on a wide array of biological processes. These small (typically 18-24 nucleotides long) non-coding RNA molecules can modulate protein expression patterns by promoting RNA degradation, inhibiting mRNA translation, and also affecting gene transcription miRs play pivotal roles in diverse processes such as development and differentiation, control of cell proliferation, stress response and metabolism. There are currently about 1100 known human miRs. The expression of many miRs was found to be altered in numerous types of human cancer, and in some cases strong evidence has been put forward in support of the conjecture that such alterations may play a causative role in tumor progression.

Mesothelioma is a tumor that occurs in the mesothelium that covers the surface of the pleura, peritoneum and pericardium that respectively envelop the organs of the chest cavity such as the rungs and heart, and abdominal organs such as the digestive tract and liver. In the case of diffuse pleural mesothelioma, chest pain is caused by invasion of the intercostal nerves on the side of the chest wall pleura, and respiratory and circulatory disorders may occur due to tumor growth and accumulation of pleural fluid in the pleura on the organ side (Takagi, Journal of Clinical and Experimental Medicine, (March Supplement), “Respiratory Diseases”, pp. 469-472, 1999). There is eventually proliferation into the adjacent mediastinal organs, progressing to direct invasion of the heart or development into the abdominal cavity by means of the diaphragm, or there may be development outside the chest cavity as a result of additional lymphatic or circulatory metastasis.

In the U.S., diffuse pleural mesothelioma is reported to occur in 3,000 persons annually, the number of cases began to increase prominently in the 1980's, and is frequently observed in men in their sixties, with the incidence in men being roughly five times that in women. According to recent reports in the U.S. and Europe, the incidence of mesothelioma is demonstrating a rapidly increasing trend, and based on epidemiological statistics from the U.K. in 1995, the number of deaths from mesothelioma is predicted to continue to increase over the next 25 years, and in the worst possible scenario, has been indicated as having a risk to the extent of accounting for 1% of all deaths among men born in the 1940's. Numerous different classifications of the clinical disease stages have been used for mesothelioma, and since the methods for classifying the disease stage used differ, previous therapeutic reports on mesothelioma have encountered difficulties when comparing the results of treatment (Nakano, Respiration, Vol. 18, No. 9, pp. 916-925, 1999). In addition, malignant mesothelioma has a causative relationship with exposure to asbestos, and this has also been demonstrated in animal experiments (Tada, Journal of Clinical and Experimental Medicine (March Supplement), “Respiratory Diseases”, pp. 406-408, 1999). Asbestos that has been inhaled into the respiratory tract reaches a location directly beneath the pleura where a tumor eventually develops due to chronic irritation for typically 20 years, and this tumor spreads in a thin layer over the entire surface of the pleura. Consequently, although malignant mesothelioma is classified as an asbestos-related disease, not all malignant mesothelioma is caused by asbestos, and well-documented exposure is only observed in about half of all patients. Malignant pleural mesothelioma (MPM) is resistant to treatment, associated with an extremely poor prognosis, and requires that countermeasures be taken immediately (Nakano, Respiration, Vol. 18, No. 9, pp. 916-925, 1999).

The prognosis for malignant mesothelioma is influenced by the stage of the disease. Surgery, when performed as part of a multimodality therapy with cytotoxic chemotherapy and radiation therapy, as well as adjuvant immunological treatments (e.g., interferon or interleukin) can be effective treatments, but only in the rare event of an early stage diagnosis.

When dealing with the possibility of a mesothelioma in the pleura or the peritoneum few differential indications should be considered. Both the pleura and the peritoneum can have secondary malignancies with primaries at different rates, hence differentiation between mesothelioma and secondary malignancy or another primary from different source is important.

Much emphasis has been placed on the discovery and characterization of a unique tumor marker. However, no marker has been identified that has adequate sensitivity or specificity to be clinically useful, although a combination of multiple markers has been shown to increase diagnostic accuracy. There is an unmet need for specific and accurate markers associated with mesothelioma, including those which would have prognostic significance in order to determine the extent of therapy necessary or reasonable for survival.

### SUMMARY OF THE INVENTION

According to some aspects of the present invention altered expression levels of SEQ ID NO: 1-77 or any combinations thereof, are indicative of mesothelioma prognosis.

According to one aspect of the invention a method for determining a prognosis for mesothelioma in a subject is provided, the method comprising:
(a) providing a biological sample from the subject;
(b) determining the expression level of a nucleic acid sequence selected from the group consisting of SEQ ID NOS: 1-16, 21-77 and sequences at least about 80% identical thereto from said sample; and
(c) comparing said expression level to a threshold expression level,
wherein the level of any of SEQ ID NOS: 1-16, 21-77 and sequences at least about 80% identical thereto compared to said threshold expression level is indicative of the prognosis of said subject.

In some embodiments the nucleic acid sequence is selected from the group consisting of SEQ ID NOS: 1-4, 11-12, 21, 22, 27-35, 45-47, 52-59, 70-72 and sequences at least about 80% identical thereto, and an increased expression level of any of said nucleic acid sequence compared to the threshold expression level is indicative of good prognosis of said subject. In other embodiments the nucleic acid sequence is selected from the group consisting of SEQ ID NOS: 5-10, 13-16, 23-26, 36-44, 48-51, 60-69, 73-77 and sequences at least about 80% identical thereto, and an increased expression level of any of said nucleic acid sequence compared to the threshold expression level is indicative of poor prognosis of said subject.

In certain embodiments the subject is a human. In certain embodiments the method is used to determine a course of treatment of the subject.

In certain embodiments the biological sample obtained from the subject is selected from the group consisting of a bodily fluid, a cell line or a tissue sample. In certain embodiments the tissue is a fresh, frozen, fixed, wax-embedded or formalin-fixed, paraffin-embedded (FFPE) tissue.

In certain embodiments the tissue is mesothelium.

According to some embodiments the expression levels are determined by a method selected from the group consisting of nucleic acid hybridization, nucleic acid amplification, or a combination thereof. According to some embodiments the nucleic acid hybridization is performed using a solid-phase nucleic acid biochip array or in situ hybridization.

According to other embodiments the nucleic acid amplification method is real-time PCR. According to some embodiments the PCR method comprises forward and reverse primers. According to some embodiments the forward primer is partially complementary to SEQ ID NOS: 1-16, 21-77, to a fragment thereof or to a sequence at least about 80% identical thereto.

According to some embodiments, the forward primer comprises a sequence selected from the group consisting of SEQ ID NOS: 78-100 and sequences at least about 80% identical thereto.

According to some embodiments, the reverse primer comprises SEQ ID NO: 124 and sequences at least about 80% identical thereto.

According to some embodiments the real-time PCR method further comprises a probe. According to some embodiments the probe is complementary to SEQ ID NOS: 1-16, 21-77 to a fragment thereof or to a sequence at least about 80% identical thereto.

According to some embodiments the probe comprises a sequence selected from the group consisting of SEQ ID NOS: 101-123 and sequences at least about 80% identical thereto.

The invention further provides a kit for prognosis of mesothelioma, said kit comprising forward and reverse primers and a probe. According to some embodiments the forward primer is partially complementary to SEQ ID NOS: 1-16, 21-77, to a fragment thereof or to a sequence at least about 80% identical thereto. According to some embodiments, the forward primer comprises a sequence selected from the group consisting of SEQ ID NOS: 78-100 and sequences at least about 80% identical thereto. According to some embodiments, the reverse primer comprises SEQ ID NO: 124 and sequences at least about 80% identical thereto. According to some embodiments the probe comprises a nucleic acid sequence that is complementary to a sequence selected from SEQ ID NO: 1-16, 21-77, to a fragment thereof or to a sequence at least about 80% identical thereto. According to some embodiments the probe comprises a sequence selected from the group consisting of SEQ ID NOS: 101-123 and sequences at least about 80% identical thereto.

According to some embodiments the kit comprises reagents for performing in situ hybridization analysis. According to some embodiments the kit comprises reagents for performing solid-phase nucleic acid biochip array.

### BRIEF DESCRIPTION OF THE DRAWINGS

FIGS. 1A-1D are Kaplan Meier plots of mesothelioma patients after surgical operation, grouped by expression levels (in units of log₂(expression)) of differentially-expressed miRs, in patients with good vs. poor prognosis. The y-axis depicts the fraction of surviving patients and the x-axis depicts months of survival.

FIG. 1A depicts the expression of hsa-miR-130b (SEQ ID NO: 9). 18 patients had expression of 9.56 units or less (solid curve) with a median survival of 12 months, and 19 patients had an expression of 9.65 or more (dashed curve) with a median survival of 4 months. p-value by log-rank=0.0009

FIG. 1B depicts the expression of hsa-miR-29c* (SEQ ID NO: 19). 18 patients had expression of less than 8.15 units (solid curve) with a median survival of 4 months, and 19 patients had an expression above 8.15 (dashed curve) with a median survival of 14 months. p-value by log-rank=0.0027

FIG. 1C depicts the expression of hsa-miR-224 (SEQ ID NO: 25). 18 patients had expression of 8.71 units or less (solid curve) with a median survival of 10 months, and 19 patients had an expression of 8.74 or more (dashed curve) with a median survival of 4 months. p-value by log-rank=0.0015

FIG. 1D depicts the expression of hsa-miR-34a (SEQ ID NO: 27). 18 patients had expression of 11.63 units or less (solid curve) with a median survival of 4 months, and 19 patients had an expression of 11.65 or more (dashed curve) with a median survival of 13 months. p-value by log-rank=0.0148

FIG. 2 Distribution of p-values calculated using the discrimination index on each of 100,000 random redistributions of expression data of miR-29c* (SEQ ID NO: 19) for the MPM patients. The y-axis depicts frequency and the x-axis depicts the discrimination index-score C. The vertical solid line presents the calculated discrimination index-score C (0.726) corresponding to miR-29c* (SEQ ID NO: 19).

### DETAILED DESCRIPTION OF THE INVENTION

The invention is based in part on the discovery that miRNA expression can serve as a novel tool for the determination of prognosis of mesothelioma.

According to some aspects of the invention, specific nucleic acid sequences (SEQ ID NOS: 1-77 and sequences at least about 80% identical thereto) may be used for the determination of prognosis of mesothelioma.

Before the present compositions and methods are disclosed and described, it is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9 and 7.0 are explicitly contemplated.

### 1. Definitions

About

As used herein, the term “about” refers to +/−10%.

Altered Expression

As used herein, the term “altered expression” encompasses over-expression, under-expression, and ectopic expression. According to some embodiments, the altered expression level is a change in a score based on a combination of expression levels of nucleic acid sequences or any combinations thereof.

Amelioration

Amelioration, as used herein, refers to a lessening of severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

Antisense

The term “antisense,” as used herein, refers to nucleotide sequences which are complementary to a specific DNA or RNA sequence. The term “antisense strand” is used in reference to a nucleic acid strand that is complementary to the “sense” strand. Antisense molecules may be produced by any method, including synthesis by ligating the gene(s) of interest in a reverse orientation to a viral promoter which permits the synthesis of a complementary strand. Once introduced into a cell, this transcribed strand combines with natural sequences produced by the cell to form duplexes. These duplexes then block either the further transcription or translation. In this manner, mutant phenotypes may be generated.

Attached

“Attached” or “immobilized” as used herein refer to a probe and a solid support and may mean that the binding between the probe and the solid support is sufficient to be stable under conditions of binding, washing, analysis, and removal. The binding may be covalent or non-covalent. Covalent bonds may be formed directly between the probe and the solid support or may be formed by a cross linker or by inclusion of a specific reactive group on either the solid support or the probe, or both. Non-covalent binding may be one or more of electrostatic, hydrophilic, and hydrophobic interactions. Included in non-covalent binding is the covalent attachment of a molecule, such as streptavidin, to the support and the non-covalent binding of a biotinylated probe to the streptavidin. Immobilization may also involve a combination of covalent and non-covalent interactions.

Biological Sample

“Biological sample” as used herein means a sample of biological tissue or fluid that comprises nucleic acids. Such samples include, but are not limited to, tissue or fluid isolated from subjects. Biological samples may also include sections of tissues such as biopsy and autopsy samples, FFPE samples, frozen sections taken for histological purposes, blood, plasma, serum, sputum, stool, tears, mucus, hair, and skin. Biological samples also include explants and primary and/or transformed cell cultures derived from animal or patient tissues. Biological samples may also be blood, a blood fraction, urine, effusions, ascitic fluid, saliva, cerebrospinal fluid, cervical secretions, vaginal secretions, endometrial secretions, gastrointestinal secretions, bronchial secretions, sputum, cell line, tissue sample, cellular content of fine needle aspiration (FNA) or secretions from the breast. A biological sample may be provided by removing a sample of cells from an animal, but can also be accomplished by using previously isolated cells (e.g., isolated by another person, at another time, and/or for another purpose), or by performing the methods described herein in vivo. Archival tissues, such as those having treatment or outcome history, may also be used. Biological samples may also be stored in RNAlater® for analysis at a later date.

Cancer

The term “cancer” is meant to include all types of cancerous growths or oncogenic processes, metastatic tissues or malignantly transformed cells, tissues, or organs, irrespective of histopathologic type or stage of invasiveness. Examples of cancers include but are nor limited to solid tumors and leukemias, including: glioblastoma, apudoma, choristoma, branchioma, malignant carcinoid syndrome, carcinoid heart disease, carcinoma (e.g., Walker, basal cell, basosquamous, Brown-Pearce, ductal, Ehrlich tumor, small cell lung, non-small cell lung (e.g., lung squamous cell carcinoma, lung adenocarcinoma and lung undifferentiated large cell carcinoma), oat cell, papillary, bronchiolar, bronchogenic, squamous cell, and transitional cell), histiocytic disorders, leukemia (e.g., B cell, mixed cell, null cell, T cell, T-cell chronic, HTLV-II-associated, lymphocytic acute, lymphocytic chronic, mast cell, and myeloid), histiocytosis malignant, Hodgkin disease, immunoproliferative small, non-Hodgkin lymphoma, plasmacytoma, reticuloendotheliosis, melanoma, chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewing sarcoma, synovioma, adenofibroma, adenolymphoma, carcinosarcoma, chordoma, craniopharyngioma, dysgerminoma, hamartoma, mesenchymoma, mesonephroma, myosarcoma, ameloblastoma, cementoma, odontoma, teratoma, thymoma, mesothelioma, trophoblastic tumor, adeno-carcinoma, adenoma, cholangioma, cholesteatoma, cylindroma, cystadenocarcinoma, cystadenoma, granulosa cell tumor, gynandroblastoma, hepatoma, hidradenoma, islet cell tumor, Leydig cell tumor, papilloma, Sertoli cell tumor, theca cell tumor, leiomyoma, leiomyosarcoma, myoblastoma, myosarcoma, rhabdomyoma, rhabdomyosarcoma, ependymoma, ganglioneuroma, glioma, medulloblastoma, meningioma, neurilemmoma, neuroblastoma, neuroepithelioma, neurofibroma, neuroma, paraganglioma, paraganglioma nonchromaffin, angiokeratoma, angiolymphoid hyperplasia with eosinophilia, angioma sclerosing, angiomatosis, glomangioma, hemangioendothelioma, hemangioma, hemangiopericytoma, hemangiosarcoma, lymphangioma, lymphangiomyoma, lymphangiosarcoma, pinealoma, carcinosarcoma, chondrosarcoma, cystosarcoma, phyllodes, fibrosarcoma, hemangiosarcoma, leimyosarcoma, leukosarcoma, liposarcoma, lymphangiosarcoma, myosarcoma, myxosarcoma, ovarian carcinoma, rhabdomyosarcoma, sarcoma (e.g., Ewing, experimental, Kaposi, and mast cell), neurofibromatosis, and cervical dysplasia, and other conditions in which cells have become immortalized or transformed.

Cancer Prognosis

A forecast or prediction of the probable course or outcome of the cancer and response to its treatment. As used herein, cancer prognosis includes distinguishing between cancer stages and subtypes, and the forecast or prediction of any one or more of the following: duration of survival of a patient susceptible to or diagnosed with a cancer, duration of recurrence-free survival, duration of progression free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis in a patient susceptible to or diagnosed with a cancer. As used herein, “prognostic for cancer” means providing a forecast or prediction of the probable course or outcome of the cancer. In some embodiments, “prognostic for cancer” comprises providing the forecast or prediction of (prognostic for) any one or more of the following: duration of survival of a patient susceptible to or diagnosed with a cancer, duration of recurrence-free survival, duration of progression free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis in a patient susceptible to or diagnosed with a cancer.

Chemotherapeutic Agent

A drug used to treat a disease, especially cancer. In relation to cancer the drugs typically target rapidly dividing cells, such as cancer cells. Non-limiting examples of chemotherapeutic agents include cisplatin, carboplatin, camptothecins, doxorubicin, cyclophosphamide, paclitaxel, etoposide, vinblastine, Actinomycin D and cloposide.

Classification

“Classification” as used herein refers to a procedure and/or algorithm in which individual items are placed into groups or classes based on quantitative information on one or more characteristics inherent in the items (referred to as traits, variables, characters, features, etc) and based on a statistical model and/or a training set of previously labeled items. According to one embodiment, classification means determination of the type of cancer.

Complement

“Complement” or “complementary” as used herein means Watson-Crick (e.g., A-T/U and C-G) or Hoogsteen base pairing between nucleotides or nucleotide analogs of nucleic acid molecules. A full complement or fully complementary may mean 100% complementary base pairing between nucleotides or nucleotide analogs of nucleic acid molecules. In some embodiments, the complementary sequence has a reverse orientation (5′-3′).

Cox Regression

As used herein “Cox regression” is a statistical technique used to determine the relationship between survival and several independent exploratory variables. This survival analysis is useful for modeling the time to a specific event based upon the value of a given covariate. Cox regression provides an estimate of a treatment on the survival rate, after adjustment of the exploratory variable. In Cox regression, the coefficient of the exploratory variable is estimated. The basic model for Cox regression produces the proportional hazard function, which can be extended through the specifications of a strata variable or time-dependent covariates.

Ct

Ct signals represent the first cycle of PCR where amplification crosses a threshold (cycle threshold) of fluorescence. Accordingly, low values of Ct represent high abundance or expression levels of the microRNA.

In some embodiments the PCR Ct signal is normalized such that the normalized Ct remains inversed from the expression level. In other embodiments the PCR Ct signal may be normalized and then inverted such that low normalized-inverted Ct represents low abundance or expression levels of the microRNA.

Detection

“Detection” means detecting the presence of a component in a sample. Detection also means detecting the absence of a component. Detection also means measuring the level of a component, either quantitatively or qualitatively.

Differential Expression

“Differential expression” means qualitative or quantitative differences in the temporal and/or cellular gene expression patterns within and among cells and tissue. Thus, a differentially expressed gene may qualitatively have its expression altered, including an activation or inactivation, in, e.g., normal versus disease tissue. Genes may be turned on or turned off in a particular state, relative to another state thus permitting comparison of two or more states. A qualitatively regulated gene may exhibit an expression pattern within a state or cell type which may be detectable by standard techniques. Some genes may be expressed in one state or cell type, but not in both. Alternatively, the difference in expression may be quantitative, e.g., in that expression is modulated, either up-regulated—resulting in an increased amount of transcript, or down-regulated—resulting in a decreased amount of transcript. The degree to which expression differs need only be large enough to quantify via standard characterization techniques such as expression arrays, quantitative reverse transcriptase PCR, northern analysis, real-time PCR, in situ hybridization and RNase protection.

Discrimination Index

As used herein, “discrimination index” is a statistical tool for survival analysis, which allows for continuous independent covariates. The discrimination index-score C, for a specific independent variable x, is a score which ranges from 0 to 1, measuring the concordance between the value of x and the survival time. The closer C is to 1, the more predictive higher values of x are for longer survival time. Conversely, C close to 0 means that lower values of x are predictive for longer survival time and C˜=0.5 means x is not correlated to the survival time.

Dose

“Dose” as used herein means a specified quantity of a pharmaceutical agent provided in a single administration. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual.

Dosage Unit

“Dosage unit” as used herein means a form in which a pharmaceutical agent is provided. In certain embodiments, a dosage unit is a vial containing lyophilized oligonucleotide. In certain embodiments, a dosage unit is a vial containing reconstituted oligonucleotide.

Expression Profile

“Expression profile” as used herein may mean a genomic expression profile, e.g., an expression profile of microRNAs. Profiles may be generated by any convenient means for determining a level of a nucleic acid sequence e.g. quantitative hybridization of microRNA, labeled microRNA, amplified microRNA, cRNA, etc., quantitative PCR, ELISA for quantitation, and the like, and allow the analysis of differential gene expression between two samples. A subject or patient tumor sample, e.g., cells or collections thereof, e.g., tissues, is assayed. Samples are collected by any convenient method, as known in the art. Nucleic acid sequences of interest are nucleic acid sequences that are found to be predictive, including the nucleic acid sequences provided above, where the expression profile may include expression data for 5, 10, 20, 25, 50, 100 or more of, including all of the listed nucleic acid sequences. The term “expression profile” may also mean measuring the abundance of the nucleic acid sequences in the measured samples.

Expression Ratio

“Expression ratio” as used herein refers to relative expression levels of two or more nucleic acids as determined by detecting the relative expression levels of the corresponding nucleic acids in a biological sample.

FDR

When performing multiple statistical tests, for example in comparing the signal between two groups in multiple data features, there is an increasingly high probability of obtaining false positive results, by random differences between the groups that can reach levels that would otherwise be considered as statistically significant. In order to limit the proportion of such false discoveries, statistical significance is defined only for data features in which the differences reached a p-value (by two-sided t-test) below a threshold, which is dependent on the number of tests performed and the distribution of p-values obtained in these tests.

Fragment

“Fragment” is used herein to indicate a non-full length part of a nucleic acid or polypeptide. Thus, a fragment is itself also a nucleic acid or polypeptide, respectively.

Gene

“Gene” as used herein may be a natural (e.g., genomic) or synthetic gene comprising transcriptional and/or translational regulatory sequences and/or a coding region and/or non-translated sequences (e.g., introns, 5′- and 3′-untranslated sequences). The coding region of a gene may be a nucleotide sequence coding for an amino acid sequence or a functional

RNA, such as tRNA, rRNA, catalytic RNA, siRNA, miRNA or antisense RNA. A gene may also be an mRNA or cDNA corresponding to the coding regions (e.g., exons and miRNA) optionally comprising 5′- or 3′-untranslated sequences linked thereto. A gene may also be an amplified nucleic acid molecule produced in vitro comprising all or a part of the coding region and/or 5′- or 3′-untranslated sequences linked thereto.

Groove Binder/Minor Groove Binder (MGB)

“Groove binder” and/or “minor groove binder” may be used interchangeably and refer to small molecules that fit into the minor groove of double-stranded DNA, typically in a sequence-specific manner. Minor groove binders may be long, flat molecules that can adopt a crescent-like shape and thus, fit snugly into the minor groove of a double helix, often displacing water. Minor groove binding molecules may typically comprise several aromatic rings connected by bonds with torsional freedom such as furan, benzene, or pyrrole rings. Minor groove binders may be antibiotics such as netropsin, distamycin, berenil, pentamidine and other aromatic diamidines, Hoechst 33258, SN 6999, aureolic anti-tumor drugs such as chromomycin and mithramycin, CC-1065, dihydrocyclopyrroloindole tripeptide (DPI₃), 1,2-dihydro-(3H)-pyrrolo[3,2-e]indole-7-carboxylate (CDPI₃), and related compounds and analogues, including those described in Nucleic Acids in Chemistry and Biology, 2d ed., Blackburn and Gait, eds., Oxford University Press, 1996, and PCT Published Application No. WO 03/078450, the contents of which are incorporated herein by reference. A minor groove binder may be a component of a primer, a probe, a hybridization tag complement, or combinations thereof. Minor groove binders may increase the Tₘ of the primer or a probe to which they are attached, allowing such primers or probes to effectively hybridize at higher temperatures.

Hazard

As used herein hazard is as used in survival analysis theory, in which hazard is the probability per time unit of an event of interest occurring at time t, given that it had not occurred before time t.

Host Cell

“Host cell” as used herein may be a naturally occurring cell or a transformed cell that may contain a vector and may support replication of the vector.

Identity

“Identical” or “identity” as used herein in the context of two or more nucleic acids or polypeptide sequences mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

In Situ Detection

“In situ detection” as used herein means the detection of expression or expression levels in the original site hereby meaning in a tissue sample such as biopsy.

Inhibit

“Inhibit” as used herein may mean prevent, suppress, repress, reduce or eliminate. Kaplan-Meier

As used herein The Kaplan-Meier method estimates the survival function from life-time data. A plot of the Kaplan-Meier estimate of the survival function is a series of horizontal steps of declining magnitude which, when a large enough sample is taken, approaches the true survival function for that population. The value of the survival function between successive distinct sampled observations is assumed to be constant. An advantage of the Kaplan-Meier curve is that the method can take into account some types of censored data, particularly censoring, which occurs if a patient withdraws from a study, i.e. is lost from the sample before the final outcome is observed. On the plot, small vertical tick-marks indicate losses, where a patient's survival time has been right-censored.

Label

“Label” as used herein means a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and other entities which can be made detectable. A label may be incorporated into nucleic acids and proteins at any position.

Logistic Regression

Logistic regression is part of a category of statistical models called generalized linear models. Logistic regression allows one to predict a discrete outcome, such as group membership, from a set of variables that may be continuous, discrete, dichotomous, or a mix of any of these. The dependent or response variable is dichotomous, for example, one of two possible types of cancer. Logistic regression models the natural log of the odds ratio, i.e. the ratio of the probability of belonging to the first group (P) over the probability of belonging to the second group (1-P), as a linear combination of the different expression levels (in log-space) and of other explaining variables. The logistic regression output can be used as a classifier by prescribing that a case or sample will be classified into the first type if P is greater than 0.5 or 50%. Alternatively, the calculated probability P can be used as a variable in other contexts such as a 1D or 2D threshold classifier.

Metastasis

“Metastasis” as used herein means the process by which cancer spreads from the place at which it first arose as a primary tumor to other locations in the body. The metastatic progression of a primary tumor reflects multiple stages, including dissociation from neighboring primary tumor cells, survival in the circulation, and growth in a secondary location.

Mismatch

“Mismatch” means a nucleobase of a first nucleic acid that is not capable of pairing with a nucleobase at a corresponding position of a second nucleic acid.

Modulation

“Modulation” as used herein means a perturbation of function or activity. In certain embodiments, modulation means an increase in gene expression. In certain embodiments, modulation means a decrease in gene expression.

Nucleic Acid

“Nucleic acid” or “oligonucleotide” or “polynucleotide” as used herein mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

A nucleic acid will generally contain phosphodiester bonds, although nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference. Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; O— and N-alkylated nucleotides, e.g. N6-methyl adenosine are suitable. The 2′-OH-group may be replaced by a group selected from H, OR, R, halo, SH, SR, NH₂, NHR, NR₂ or CN, wherein R is C₁-C₆ alkyl, alkenyl or alkynyl and halo is F, Cl, Br or I. Modified nucleotides also include nucleotides conjugated with cholesterol through, e.g., a hydroxyprolinol linkage as described in Krutzfeldt et al., Nature 438:685-689 (2005) and Soutschek et al., Nature 432:173-178 (2004), which are incorporated herein by reference. Modifications of the ribose-phosphate backbone may be done for a variety of reasons, e.g., to increase the stability and half-life of such molecules in physiological environments, to enhance diffusion across cell membranes, or as probes on a biochip. The backbone modification may also enhance resistance to degradation, such as in the harsh endocytic environment of cells. The backbone modification may also reduce nucleic acid clearance by hepatocytes, such as in the liver. Mixtures of naturally occurring nucleic acids and analogs may be made; alternatively, mixtures of different nucleic acid analogs, and mixtures of naturally occurring nucleic acids and analogs may be made.

Overall Survival Time

“Overall survival time” or “survival time”, as used herein means the time period for which a subject survives after diagnosis of or treatment for a disease. In certain embodiments, the disease is cancer.

Partially Complementary

As used herein, “partially complementary” refers to less than 100% complementary base pairing between nucleotides or nucleotide analogs of nucleic acid molecules. In some embodiments the base-paring spans only a specific section of the partially complementary nucleic acid molecules.

Pharmaceutical Agent

Pharmaceutical agent as used herein means a substance that provides a therapeutic effect when administered to a subject. “Pharmaceutical composition” means a mixture of substances suitable for administering to an individual that includes a pharmaceutical agent. For example, a pharmaceutical composition may comprise a modified oligonucleotide and a sterile aqueous solution. “Active pharmaceutical ingredient” means the substance in a pharmaceutical composition that provides a desired effect.

Prevention

Prevention as used herein means delaying or forestalling the onset or development or progression of a condition or disease for a period of time, including weeks, months, or years.

Progression-Free Survival

“Progression-free survival” means the fraction of subjects having a disease condition which survived at a given time without the disease getting worse. In certain embodiments, progression-free survival is assessed by staging or scoring the disease. In certain embodiments progression-free survival of a subject having cancer is assessed by evaluating tumor size, tumor number, and/or metastasis.

Probe

“Probe” as used herein means an oligonucleotide capable of binding to a target nucleic acid of complementary sequence through one or more types of chemical bonds, usually through complementary base pairing, usually through hydrogen bond formation. Probes may bind target sequences lacking complete complementarity with the probe sequence depending upon the stringency of the hybridization conditions. There may be any number of base pair mismatches which will interfere with hybridization between the target sequence and the single stranded nucleic acids described herein. However, if the number of mutations is so great that no hybridization can occur under even the least stringent of hybridization conditions, the sequence is not a complementary target sequence. A probe may be single stranded or partially single and partially double stranded. The strandedness of the probe is dictated by the structure, composition, and properties of the target sequence. Probes may be directly labeled or indirectly labeled such as with biotin to which a streptavidin complex may later bind.

Promoter

“Promoter” as used herein means a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents.

Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

Q-Value

The q-value of an individual hypothesis test is the minimum FDR at which the test may be called significant. q-value is the minimum positive FDR that can occur when rejecting a statistic with the given value for the set of nested significance regions.

Reference Expression Profile

As used herein the term “reference expression profile” means a value that statistically correlates to a particular outcome when compared to an assay result. In preferred embodiments the reference value is determined from statistical analysis of studies that compare microRNA expression with known clinical outcomes. The reference value may be a threshold score value or a cutoff score value. Typically a reference value will be a threshold above which one outcome is more probable and below which an alternative threshold is more probable.

Sensitivity

“sensitivity” used herein may mean a statistical measure of how well a binary classification test correctly identifies a condition, for example how frequently it correctly classifies a cancer into the correct type out of two possible types. The sensitivity for class A is the proportion of cases that are determined to belong to class “A” by the test out of the cases that are in class “A”, as determined by some absolute or gold standard.

Specificity

“Specificity” used herein may mean a statistical measure of how well a binary classification test correctly identifies a condition, for example how frequently it correctly classifies a cancer into the correct type out of two possible types. The specificity for class A is the proportion of cases that are determined to belong to class “not A” by the test out of the cases that are in class “not A”, as determined by some absolute or gold standard.

Side Effect

Side effect as used herein means a physiological response attributable to a treatment other than desired effects.

Selectable Marker

“Selectable marker” as used herein means any gene which confers a phenotype on a host cell in which it is expressed to facilitate the identification and/or selection of cells which are transfected or transformed with a genetic construct. Representative examples of selectable markers include the ampicillin-resistance gene (Amp^{r}), tetracycline-resistance gene (Tc^{r}), bacterial kanamycin-resistance gene (Kan^{r}), zeocin resistance gene, the AURI-C gene which confers resistance to the antibiotic aureobasidin A, phosphinothricin-resistance gene, neomycin phosphotransferase gene (nptII), hygromycin-resistance gene, beta-glucuronidase (GUS) gene, chloramphenicol acetyltransferase (CAT) gene, green fluorescent protein (GFP)-encoding gene and luciferase gene.

Stringent Hybridization Conditions

“Stringent hybridization conditions” as used herein mean conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic acid concentration) at which 50% of the probes complementary to the target hybridize to the target sequence at equilibrium (as the target sequences are present in excess, at Tₘ, 50% of the probes are occupied at equilibrium).

Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

Substantially Complementary

“Substantially complementary” as used herein means that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides, or that the two sequences hybridize under stringent hybridization conditions.

Substantially Identical

“Substantially identical” as used herein means that a first and a second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

Subject

As used herein, the term “subject” refers to a human or non-human animal selected for treatment or therapy. The methods of the present invention are preferably applied to human subjects. “Subject in need thereof” refers to a subject identified as in need of a therapy or treatment. In certain embodiments, a subject is in need of treatment for mesothelioma. In such embodiments, a subject has one or more clinical indications of mesothelioma or is at risk for developing mesothelioma.

Target Nucleic Acid

“Target nucleic acid” as used herein means a nucleic acid or variant thereof that may be bound by another nucleic acid. A target nucleic acid may be a DNA sequence. The target nucleic acid may be RNA. The target nucleic acid may comprise a mRNA, tRNA, shRNA, siRNA or Piwi-interacting RNA, or a pri-miRNA, pre-miRNA, miRNA, or anti-miRNA.

The target nucleic acid may comprise a target miRNA binding site or a variant thereof. One or more probes may bind the target nucleic acid. The target binding site may comprise 5-100 or 10-60 nucleotides. The target binding site may comprise a total of 5, 6, 7, 8, 9, 10, 11, 12, 13, 14, 15,16, 17, 18, 19, 20, 21, 22, 23, 24, 25, 26, 27, 28, 29, 30-40, 40-50, 50-60, 61, 62 or 63 nucleotides. The target site sequence may comprise at least 5 nucleotides of the sequence of a target miRNA binding site disclosed in U.S. patent application Ser. Nos. 11/384,049, 11/418,870 or 11/429,720, the contents of which are incorporated herein.

Therapy

“Therapy” as used herein means a disease treatment method. In certain embodiments, therapy includes, but is not limited to, tyrosine kinase inhibition therapy, chemotherapy, surgical resection, transplant, radiation therapy, “gene therapy”, immunotherapy, and/or chemoembolization. “Therapeutic agent” means a pharmaceutical agent used for the cure, amelioration or prevention of a disease. “Recommended therapy” means a treatment recommended by a medical professional for the treatment, amelioration, or prevention of a disease.

Therapeutically Effective Amount

“Therapeutically effective amount” or “therapeutically efficient” used herein as to a drug dosage, refer to dosage that provides the specific pharmacological response for which the drug is administered in a significant number of subjects in need of such treatment. The “therapeutically effective amount” may vary according, for example, the physical condition of the patient, the age of the patient and the severity of the disease.

Threshold Expression Level

As used herein, the phrase “threshold expression level” refers to a reference expression value. Measured values are compared to a corresponding threshold expression level to determine the prognosis of a subject.

Tissue Sample

As used herein, a tissue sample is tissue obtained from a tissue biopsy using methods well known to those of ordinary skill in the related medical arts. The phrase “suspected of being cancerous” as used herein means a cancer tissue sample believed by one of ordinary skill in the medical arts to contain cancerous cells. Methods for obtaining the sample from the biopsy include gross apportioning of a mass, microdissection, laser-based microdissection, or other art-known cell-separation methods.

Treat

“Treat” or “treating” used herein when referring to protection of a subject from a condition may mean preventing, suppressing, repressing, or eliminating the condition. Preventing the condition involves administering a composition described herein to a subject prior to onset of the condition. Suppressing the condition involves administering the composition to a subject after induction of the condition but before its clinical appearance. Repressing the condition involves administering the composition to a subject after clinical appearance of the condition such that the condition is reduced or prevented from worsening. Elimination of the condition involves administering the composition to a subject after clinical appearance of the condition such that the subject no longer suffers from the condition.

Unit Dosage Form

“Unit dosage form,” used herein may refer to a physically discrete unit suitable as a unitary dosage for a human or animal subject. Each unit may contain a predetermined quantity of a composition described herein, calculated in an amount sufficient to produce a desired effect in association with a pharmaceutically acceptable diluent, carrier or vehicle. The specifications for a unit dosage form may depend on the particular composition employed and the effect to be achieved, and the pharmacodynamics associated with the composition in the host.

Variant

“Variant” as used herein referring to a nucleic acid means (i) a portion of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequence substantially identical thereto.

Vector

“Vector” as used herein means a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

Wild Type

As used herein, the term “wild type” sequence refers to a coding, a non-coding or an interface sequence which is an allelic form of sequence that performs the natural or normal function for that sequence. Wild type sequences include multiple allelic forms of a cognate sequence, for example, multiple alleles of a wild type sequence may encode silent or conservative changes to the protein sequence that a coding sequence encodes.

1D/2D Threshold Classifier

“1D/2D threshold classifier” used herein may mean an algorithm for classifying a case or sample such as a cancer sample into one of two possible types such as two types of cancer or two types of prognosis (e.g. good and poor). For a 1D threshold classifier, the decision is based on one variable and one predetermined threshold value; the sample is assigned to one class if the variable exceeds the threshold and to the other class if the variable is less than the threshold. A 2D threshold classifier is an algorithm for classifying into one of two types based on the values of two variables. A score may be calculated as a function (usually a continuous function) of the two variables; the decision is then reached by comparing the score to the predetermined threshold, similar to the 1D threshold classifier.

2. MicroRNAs and their Processing

A gene coding for a microRNA (miRNA) may be transcribed leading to production of an miRNA precursor known as the pri-miRNA. The pri-miRNA may be part of a polycistronic RNA comprising multiple pri-miRNAs. The pri-miRNA may form a hairpin structure with a stem and loop. The stem may comprise mismatched bases.

The hairpin structure of the pri-miRNA may be recognized by Drosha, which is an RNase III endonuclease. Drosha may recognize terminal loops in the pri-miRNA and cleave approximately two helical turns into the stem to produce a 60-70 nucleotide precursor known as the pre-miRNA. Drosha may cleave the pri-miRNA with a staggered cut typical of RNase III endonucleases yielding a pre-miRNA stem loop with a 5′ phosphate and ˜2 nucleotide 3′ overhang. Approximately one helical turn of the stem (˜10 nucleotides) extending beyond the Drosha cleavage site may be essential for efficient processing. The pre-miRNA may then be actively transported from the nucleus to the cytoplasm by Ran-GTP and the export receptor Ex-portin-5.

The pre-miRNA may be recognized by Dicer, which is also an RNase III endonuclease. Dicer may recognize the double-stranded stem of the pre-miRNA. Dicer may also recognize the 5′ phosphate and 3′ overhang at the base of the stem loop. Dicer may cleave off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. MiRNA* sequences may be found in libraries of cloned miRNAs but typically at lower frequency than the miRNAs.

Although initially present as a double-stranded species with miRNA*, the miRNA may eventually become incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repression or activation), and which strand of the miRNA/miRNA* duplex is loaded in to the RISC.

When the miRNA strand of the miRNA:miRNA* duplex is loaded into the RISC, the miRNA* may be removed and degraded. The strand of the miRNA:miRNA* duplex that is loaded into the RISC may be the strand whose 5′ end is less tightly paired. In cases where both ends of the miRNA:miRNA* have roughly equivalent 5′ pairing, both miRNA and miRNA* may have gene silencing activity.

The RISC may identify target nucleic acids based on high levels of complementarity between the miRNA and the mRNA, especially by nucleotides 2-7 of the miRNA. Only one case has been reported in animals where the interaction between the miRNA and its target was along the entire length of the miRNA. This was shown for mir-196 and Hox B8 and it was further shown that mir-196 mediates the cleavage of the Hox B8 mRNA (Yekta et al 2004, Science 304-594). Otherwise, such interactions are known only in plants (Bartel & Bartel 2003, Plant Physiol 132-709).

A number of studies have studied the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85).

Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al (2005, Nat Genet 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut may be between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and the binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

### 3. Nucleic Acids

Nucleic acids are provided herein. The nucleic acids comprise the sequence of SEQ ID NOS: 1-124, or variants thereof. The variant may be a complement of the referenced nucleotide sequence. The variant may also be a nucleotide sequence that is substantially identical to the referenced nucleotide sequence or the complement thereof. The variant may also be a nucleotide sequence which hybridizes under stringent conditions to the referenced nucleotide sequence, complements thereof, or nucleotide sequences substantially identical thereto.

The nucleic acid may have a length of from 10 to 250 nucleotides. The nucleic acid may have a length of at least 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 125, 150, 175, 200 or 250 nucleotides. The nucleic acid may be synthesized or expressed in a cell (in vitro or in vivo) using a synthetic gene described herein. The nucleic acid may be synthesized as a single strand molecule and hybridized to a substantially complementary nucleic acid to form a duplex. The nucleic acid may be introduced to a cell, tissue or organ in a single- or double-stranded form or capable of being expressed by a synthetic gene using methods well known to those skilled in the art, including as described in U.S. Pat. No. 6,506,559 which is incorporated by reference.

TABLE 1
Nucleic acid sequences associated with survival of mesothelioma patients

| | miR SEQ | Hairpin SEQ |
|---|---|---|
| miR name | ID NO. | ID NO. |
| hsa-miR-378 | 1 | 2 |
| hsa-miR-342-5p | 3 | 4 |
| hsa-miR-886-3p | 5 | 6 |
| hsa-miR-152 | 7 | 8 |
| hsa-miR-130b | 9 | 10 |
| hsa-miR-140-3p | 11 | 12 |
| hsa-miR-151-3p | 13 | 14 |
| hsa-miR-320a | 15 | 16 |
| hsa-miR-21* | 17 | 18 |
| hsa-miR-29c* | 19 | 20 |
| hsa-miR-451 | 21 | 22 |
| hsa-miR-21 | 23 | 24 |
| hsa-miR-224 | 25 | 26 |
| hsa-miR-34a | 27 | 28 |
| hsa-miR-29c | 29 | 20 |
| hsa-miR-26b | 30 | 52 |
| hsa-let-7g | 31 | 53 |
| hsa-miR-106b | 32 | 54 |
| hsa-miR-101 | 33 | 55, 56 |
| hsa-miR-30e | 34 | 57 |
| hsa-miR-29b | 35 | 58, 59 |
| hsa-miR-92b* | 36 | 60 |
| hsa-miR-324-5p | 37 | 61 |
| hsa-miR-181b | 38 | 62, 63 |
| hsa-miR-424 | 39 | 64 |
| hsa-miR-25 | 40 | 65 |
| hsa-miR-149 | 41 | 66 |
| hsa-miR-92a | 42 | 67, 68 |
| hsa-miR-193b | 43 | 69 |
| hsa-miR-92b | 44 | 60 |
| hsa-miR-200b | 45 | 70 |
| hsa-miR-200a | 46 | 71 |
| hsa-miR-148a | 47 | 72 |
| hsa-miR-138 | 48 | 73, 74 |
| hsa-miR-27a | 49 | 75 |
| hsa-miR-23a | 50 | 76 |
| hsa-miR-18a | 51 | 77 |

3a. Nucleic Acid Complexes

The nucleic acid may further comprise one or more of the following: a peptide, a protein, a RNA-DNA hybrid, an antibody, an antibody fragment, a Fab fragment, and an aptamer.

3b. Pri-miRNA

The nucleic acid may comprise a sequence of a pri-miRNA or a variant thereof. The pri-miRNA sequence may comprise from 45-30,000, 50-25,000, 100-20,000, 1,000-1,500 or 80-100 nucleotides. The sequence of the pri-miRNA may comprise a pre-miRNA, miRNA and miRNA*, as set forth herein, and variants thereof.

The pri-miRNA may form a hairpin structure. The hairpin may comprise a first and a second nucleic acid sequence that are substantially complimentary. The first and second nucleic acid sequence may be from 37-50 nucleotides. The first and second nucleic acid sequence may be separated by a third sequence of from 8-12 nucleotides. The hairpin structure may have a free energy of less than −25 Kcal/mole, as calculated by the Vienna algorithm, with default parameters as described in Hofacker et al., Monatshefte f. Chemie 125: 167-188 (1994), the contents of which are incorporated herein. The hairpin may comprise a terminal loop of 4-20, 8-12 or 10 nucleotides. The pri-miRNA may comprise at least 19% adenosine nucleotides, at least 16% cytosine nucleotides, at least 23% thymine nucleotides and at least 19% guanine nucleotides.

3c. Pre-miRNA

The nucleic acid may also comprise a sequence of a pre-miRNA or a variant thereof. The pre-miRNA sequence may comprise from 45-90, 60-80 or 60-70 nucleotides. The sequence of the pre-miRNA may comprise a miRNA and a miRNA* as set forth herein. The sequence of the pre-miRNA may also be that of a pri-miRNA excluding from 0-160 nucleotides from the 5′ and 3′ ends of the pri-miRNA. The sequence of the pre-miRNA may comprise the sequence of SEQ ID NOS: 1-77, or variants thereof.

3d. miRNA

The nucleic acid may also comprise a sequence of a miRNA (including miRNA*) or a variant thereof. The miRNA sequence may comprise from 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA. The sequence of the miRNA may comprise the sequence of SEQ ID NOS: 1, 3, 5, 7, 9, 11, 13, 15, 17, 19, 21, 23, 25, 27, 29-51; or variants thereof.

3e. Anti-miRNA

The nucleic acid may also comprise a sequence of an anti-miRNA capable of blocking the activity of a miRNA or miRNA*, such as by binding to the pri-miRNA, pre-miRNA, miRNA or miRNA* (e.g. antisense or RNA silencing), or by binding to the target binding site. The anti-miRNA may comprise a total of 5-100 or 10-60 nucleotides. The anti-miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the anti-miRNA may comprise (a) at least 5 nucleotides that are substantially identical or complimentary to the 5′ of a miRNA and at least 5-12 nucleotides that are substantially complimentary to the flanking regions of the target site from the 5′ end of the miRNA, or (b) at least 5-12 nucleotides that are substantially identical or complimentary to the 3′ of a miRNA and at least 5 nucleotide that are substantially complimentary to the flanking region of the target site from the 3′ end of the miRNA.

3f. microRNA Binding Site of Target

The nucleic acid may also comprise a sequence of a target binding site or a variant thereof. The target site sequence may comprise a total of 5-100 or 10-60 nucleotides. The target site sequence may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62 or 63 nucleotides.

### 4. Synthetic Gene

A synthetic gene is also provided comprising a nucleic acid described herein operably linked to a transcriptional and/or translational regulatory sequence. The synthetic gene may be capable of modifying the expression of a target gene with a binding site for a nucleic acid described herein. Expression of the target gene may be modified in a cell, tissue or organ. The synthetic gene may be synthesized or derived from naturally-occurring genes by standard recombinant techniques. The synthetic gene may also comprise terminators at the 3′-end of the transcriptional unit of the synthetic gene sequence. The synthetic gene may also comprise a selectable marker.

### 5. Vector

A vector is also provided comprising a synthetic gene described herein. The vector may be an expression vector. An expression vector may comprise additional elements. For example, the expression vector may have two replication systems allowing it to be maintained in two organisms, e.g., in one host cell for expression and in a second host cell (e.g., bacteria) for cloning and amplification. For integrating expression vectors, the expression vector may contain at least one sequence homologous to the host cell genome, and preferably two homologous sequences which flank the expression construct. The integrating vector may be directed to a specific locus in the host cell by selecting the appropriate homologous sequence for inclusion in the vector. The vector may also comprise a selectable marker gene to allow the selection of transformed host cells.

### 6. Host Cell

A host cell is also provided comprising a vector, synthetic gene or nucleic acid described herein. The cell may be a bacterial, fungal, plant, insect or animal cell. For example, the host cell line may be DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/0 (mouse myeloma), P3x63-Ag3.653 (mouse myeloma), BFA-1c1BPT (bovine endothelial cells), RAJI (human lymphocyte) and 293 (human kidney). Host cell lines may be available from commercial services, the American Tissue Culture Collection or from published literature.

### 7. Probes

A probe is provided herein. A probe may comprise a nucleic acid. The probe may have a length of from 8 to 500, 10 to 100 or 20 to 60 nucleotides. The probe may also have a length of at least 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, 100, 120, 140, 160, 180, 200, 220, 240, 260, 280 or 300 nucleotides. The probe may comprise a nucleic acid of 18-25 nucleotides.

A probe may be capable of binding to a target nucleic acid of complementary sequence through one or more types of chemical bonds, usually through complementary base pairing, usually through hydrogen bond formation. Probes may bind target sequences lacking complete complementarity with the probe sequence depending upon the stringency of the hybridization conditions. A probe may be single stranded or partially single and partially double stranded. The strandedness of the probe is dictated by the structure, composition, and properties of the target sequence. Probes may be directly labeled or indirectly labeled.

The probe may be a test probe. The test probe may comprise a nucleic acid sequence that is complementary to a miRNA, a miRNA*, a pre-miRNA, or a pri-miRNA.

The probe may further comprise a linker. The linker may be 10-60 nucleotides in length. The linker may be 20-27 nucleotides in length. The linker may be of sufficient length to allow the probe to be a total length of 45-60 nucleotides. The linker may not be capable of forming a stable secondary structure, or may not be capable of folding on itself, or may not be capable of folding on a non-linker portion of a nucleic acid contained in the probe. The sequence of the linker may not appear in the genome of the animal from which the probe non-linker nucleic acid is derived.

### 8. Reverse Transcription

Target sequences of a cDNA may be generated by reverse transcription of the target RNA. Methods for generating cDNA may be reverse transcribing polyadenylated RNA or alternatively, RNA with a ligated adaptor sequence.

The RNA may be ligated to an adapter sequence prior to reverse transcription. A ligation reaction may be performed by T4 RNA ligase to ligate an adaptor sequence at the 3′ end of the RNA. Reverse transcription (RT) reaction may then be performed using a primer comprising a sequence that is complementary to the 3′ end of the adaptor sequence.

Polyadenylated RNA may be used in a reverse transcription (RT) reaction using a poly(T) primer comprising a 5′ adaptor sequence. The poly(T) sequence may comprise 8, 9, 10, 11, 12, 13, or 14 consecutive thymines.

The reverse transcript of the RNA may be amplified by real time PCR, using a specific forward primer comprising at least 15 nucleic acids complementary to the target nucleic acid and a 5′ tail sequence; a reverse primer that is complementary to the 3′ end of the adaptor sequence; and a probe comprising at least 8 nucleic acids complementary to the target nucleic acid. The probe may be partially complementary to the 5′ end of the adaptor sequence.

Methods of amplifying target nucleic acids are described herein. The amplification may be by a method comprising PCR. The first cycles of the PCR reaction may have an annealing temp of 56° C., 57° C., 58° C., 59° C., or 60° C. The first cycles may comprise 1-10 cycles. The remaining cycles of the PCR reaction may be 60° C. The remaining cycles may comprise 2-40 cycles. The annealing temperature may cause the PCR to be more sensitive. The PCR may generate longer products that can serve as higher stringency PCR templates.

The PCR reaction may comprise a forward primer. The forward primer may comprise 15, 16, 17, 18, 19, 20, or 21 nucleotides identical to the target nucleic acid.

The 3′ end of the forward primer may be sensitive to differences in sequence between a target nucleic acid and a sibling nucleic acid.

The forward primer may also comprise a 5′ overhanging tail. The 5′ tail may increase the melting temperature of the forward primer. The sequence of the 5′ tail may comprise a sequence that is non-identical to the genome of the animal from which the target nucleic acid is isolated. The sequence of the 5′ tail may also be synthetic. The 5′ tail may comprise 8, 9, 10, 11, 12, 13, 14, 15, or 16 nucleotides.

The PCR reaction may comprise a reverse primer. The reverse primer may be complementary to a target nucleic acid. The reverse primer may also comprise a sequence complementary to an adaptor sequence. The sequence complementary to an adaptor sequence may comprise 12-24 nucleotides.

### 9. Biochip

A biochip is also provided. The biochip may comprise a solid substrate comprising an attached probe or plurality of probes described herein. The probes may be capable of hybridizing to a target sequence under stringent hybridization conditions. The probes may be attached at spatially defined locations on the substrate. More than one probe per target sequence may be used, with either overlapping probes or probes to different sections of a particular target sequence. The probes may be capable of hybridizing to target sequences associated with a single disorder appreciated by those in the art. The probes may either be synthesized first, with subsequent attachment to the biochip, or may be directly synthesized on the biochip.

The solid substrate may be a material that may be modified to contain discrete individual sites appropriate for the attachment or association of the probes and is amenable to at least one detection method. Representative examples of substrate materials include glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses and plastics. The substrates may allow optical detection without appreciably fluorescing.

The substrate may be planar, although other configurations of substrates may be used as well. For example, probes may be placed on the inside surface of a tube, for flow-through sample analysis to minimize sample volume. Similarly, the substrate may be flexible, such as flexible foam, including closed cell foams made of particular plastics.

The substrate of the biochip and the probe may be derivatized with chemical functional groups for subsequent attachment of the two. For example, the biochip may be derivatized with a chemical functional group including, but not limited to, amino groups, carboxyl groups, oxo groups or thiol groups. Using these functional groups, the probes may be attached using functional groups on the probes either directly or indirectly using a linker

The probes may be attached to the solid support by either the 5′ terminus, 3′ terminus, or via an internal nucleotide.

The probe may also be attached to the solid support non-covalently. For example, biotinylated oligonucleotides can be made, which may bind to surfaces covalently coated with streptavidin, resulting in attachment. Alternatively, probes may be synthesized on the surface using techniques such as photopolymerization and photolithography.

### 10. Diagnostics

A method of diagnosis is also provided. The method comprises detecting a differential expression level of mesothelioma-associated nucleic acids in a biological sample. The sample may be derived from a patient. Diagnosis of a cancer state, and its histological type, in a patient may allow for prognosis and selection of therapeutic strategy. Further, the developmental stage of cells may be classified by determining temporarily expressed cancer-associated nucleic acids.

In situ hybridization of labeled probes to tissue arrays may be performed. When comparing the fingerprints between an individual and a standard, the skilled artisan can make a diagnosis, a prognosis, or a prediction based on the findings. It is further understood that the nucleic acid sequences which indicate the diagnosis may differ from those which indicate the prognosis and molecular profiling of the condition of the cells or exosomes may lead to distinctions between responsive or refractory conditions or may be predictive of outcomes.

### 11. Kits

A kit is also provided and may comprise a nucleic acid described herein together with any or all of the following: assay reagents, buffers, probes and/or primers, and sterile saline or another pharmaceutically acceptable emulsion and suspension base. In addition, the kits may include instructional materials containing directions (e.g., protocols) for the practice of the methods described herein.

For example, the kit may be used for the amplification, detection, identification or quantification of a target nucleic acid sequence. The kit may comprise a poly(T) primer, a forward primer, a reverse primer, and a probe.

Any of the compositions described herein may be comprised in a kit. In a non-limiting example, reagents for isolating miRNA, labeling miRNA, and/or evaluating a miRNA population using an array are included in a kit. The kit may further include reagents for creating or synthesizing miRNA probes. The kits will thus comprise, in suitable container means, an enzyme for labeling the miRNA by incorporating labeled nucleotide or unlabeled nucleotides that are subsequently labeled. It may also include one or more buffers, such as reaction buffer, labeling buffer, washing buffer, or a hybridization buffer, compounds for preparing the miRNA probes, components for in situ hybridization and components for isolating miRNA. Other kits of the invention may include components for making a nucleic acid array comprising miRNA, and thus, may include, for example, a solid support.

The following examples are presented in order to more fully illustrate some embodiments of the invention. They should, in no way be construed, however, as limiting the broad scope of the invention.

### EXAMPLES

### Example 1

### Materials and Methods

### Biological Samples

Thirty seven mesothelioma tumors specimens were obtained under the auspices of IRB approved tissue procurement protocols from the Karmanos Cancer Institute (KCI), Detroit Mich. (1997-2005). Specimens were obtained fresh from the operating room during cytoreductive surgery or biopsy for diagnosis and immediately snap frozen in liquid nitrogen and stored at −80° C. All specimens had immunohistochemical evidence of mesothelioma with positive staining for WT1, cytokeratins, calretinin, and the absence of staining for CEA and B72.3. Demographics including age, sex, stage, mesothelioma histology, time to progression from surgery, time to death from surgery, were recorded and current as of September 2008. Demographics of the patients from which the specimens were obtained are detailed in table 2 below.

TABLE 2
Patient demographics

| | |
|---|---|
| Age | 63 + 1(43-78) yrs. |
| Sex | 9 Female, 28 Male (76%) |
| Stage | I/II: 10 (37%); |
| | III/IV: 27 |
| Histology | 23 Epithelial (62%); |
| | 14 Biphasic or Sarcomatoid |
| Cytoreductive | 34 Y (92%); 3 N |
| Surgery | |

### Microarray Platform

Total RNA was extracted from the specimens using the mirVANA microRNA isolation kit (Ambion). ˜900 DNA oligonucleotide probes representing microRNAs were spotted in triplicate on coated microarray slides (Nexterion® Slide E, Schott, Mainz, Germany).

3-5 μg of total RNA were labeled by ligation of an RNA-linker, p-rCrU-Cy/dye (Dharmacon, Lafayette, Colo.; Cy3 or Cy5) to the 3′ end. Slides were incubated with the labeled RNA for 12-16 hr at 42° C. and then washed twice. Arrays were scanned at a resolution of 10 μm, and images were analyzed using SpotReader software (Niles Scientific, Portola Valley, Calif.). Microarray spots were combined and signals normalized.

### Example 2

### Results and Analysis

The mean follow-up for the 37 patients was 1.48 years. Among 27 patients who died of MPM, mean survival was 1.09 years, while amongst the 10 patients who survived or were censored mean follow-up was 2.54 years. Eleven patients remained MPM recurrence-free for one year, while 24 suffered recurrence within a year, with two deaths from other causes within a year. Patients were grouped by their retrospective prognosis wherein “poor prognosis” was defined as recurrence within 12 months, and “good prognosis” was defined as a time to recurrence greater than or equal to 12 months, for patients in whom progression was documented by radiographic or histological evidence of progression of disease.

microRNA expression levels were log-transformed (base 2) and then normalized by fitting to a second-degree polynomial and then transformed back to linear scale. Only microRNAs which had a median signal higher than signal background levels (normalized fluorescence signal of ˜300) in at least one of the two prognosis-groups were tested.

The expression levels of each microRNA was compared between the two prognosis-groups using ranksum. microRNAs with ranksum p-value lower than 0.05 and fold change higher than 1.5 between prognosis-groups are presented in Table 3, divided into highly expressed (upregulated) microRNAs in the good-prognosis group vs. highly expressed (upregulated) microRNAs in the poor-prognosis group. Q-value calculated using Storey.

TABLE 3
microRNA differential expression between good- and poor-prognosis (non-
censored) MPM patients. Fold-change is the ratio between the median expression (in
fluorescence units) of the microRNAs within the good and poor prognosis groups.

| | | | Normalized median | | | | |
|---|---|---|---|---|---|---|---|
| | | | expression | | | | |
| | | SEQ | good- | poor- | | | |
| | | ID | prognosis | prognosis | Fold | | |
| microRNA | | NO: | group | group | change | p-value | q-value |
| Up | hsa-miR-451 | 21 | 8500 | 3800 | 2.25 | 0.024 | 0.492 |
| regulated | hsa-miR-29c* | 19 | 480 | 230 | 2.07 | 0.001 | 0.086 |
| in good- | hsa-miR-378 | 1 | 2200 | 1100 | 2.00 | 0.049 | 0.531 |
| prognosis | hsa-miR-342-5p | 3 | 440 | 250 | 1.74 | 0.034 | 0.491 |
| group | | | | | | | |
| Up | hsa-miR-21* | 17 | 510 | 1500 | 2.90 | 0.001 | 0.113 |
| regulated | hsa-miR-886-3p | 5 | 1400 | 3700 | 2.58 | 0.004 | 0.138 |
| in poor- | hsa-miR-21 | 23 | 540 | 960 | 1.77 | 0.002 | 0.123 |
| prognosis | hsa-miR-152 | 7 | 1700 | 2700 | 1.60 | 0.005 | 0.157 |
| group | hsa-miR-130b | 9 | 620 | 960 | 1.53 | 0.001 | 0.094 |

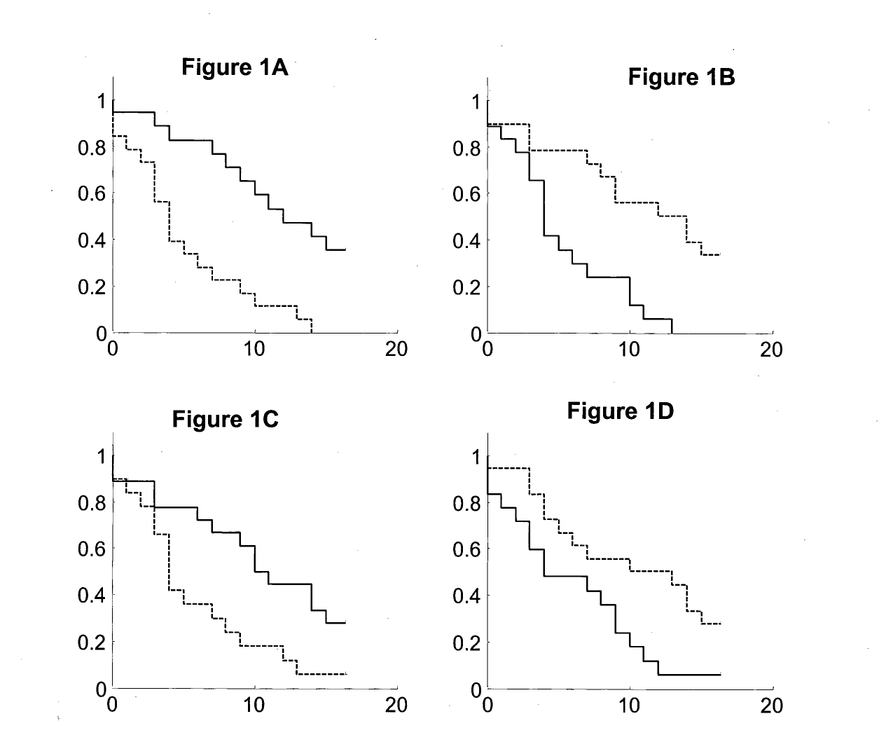

The results were also analysed by the Kaplan-Meier method, comparing between patients with higher than median miR-expression and patients with miR-expression below median. As indicated in FIG. 1, hsa-miR-130b (SEQ ID NO: 9), hsa-miR-29c* (SEQ ID NO: 19), hsa-miR-224 (SEQ ID NO: 25) and hsa-miR-34a (SEQ ID NO: 27) significantly differentiate between patients with good- and poor-prognosis, such that high expression of each of hsa-miR-29c* (SEQ ID NO: 19) and hsa-miR-34a (SEQ ID NO: 27) is associated with good prognosis, and high expression of each of hsa-miR-130b (SEQ ID NO: 9) and hsa-miR-224 (SEQ ID NO: 25) is associated with poor prognosis.

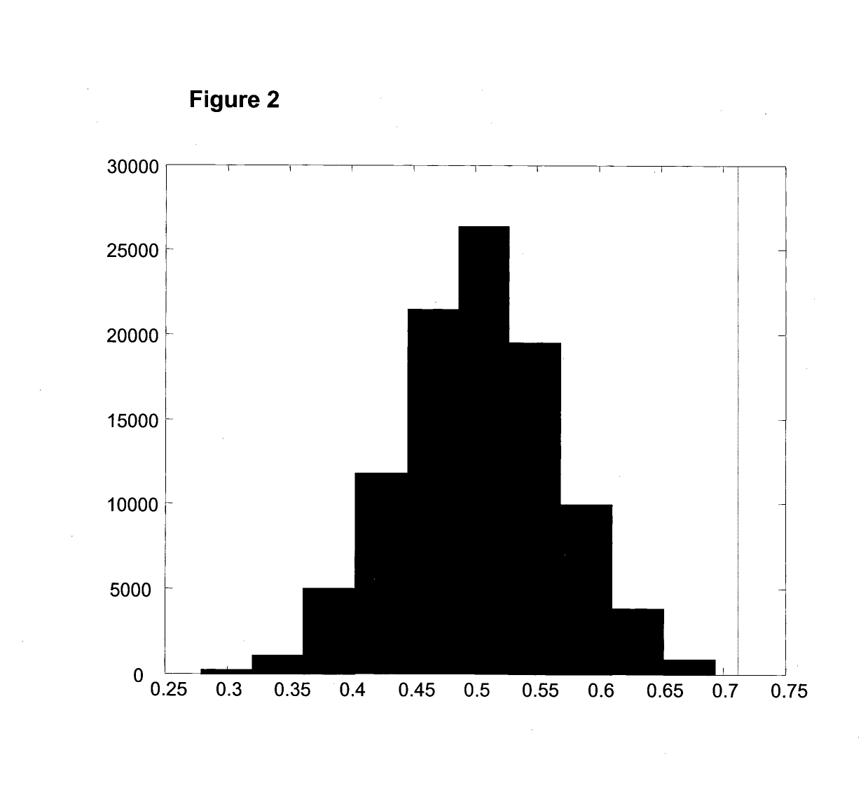

Association of microRNA expression and prognosis was further examined by calculating the discriminating index corresponding to each expressed microRNA, and comparing its score to that of other microRNAs. hsa-miR-29c* (SEQ ID NO: 19) was found to have the most significant association between expression and survival, according to the discriminating index method. A histogram of the distribution created by 100,000 random reallocations of this microRNA is presented in FIG. 2. The distribution is nearly normal, with a mean of 0.501 and a standard deviation of 0.062. Using this distribution as a reference, the p-value of other expressed microRNAs was obtained by the relative ranking of their discriminating index-score C within this envelope. The five most significant microRNAs are shown in Table 4.

TABLE 4
microRNA expression as a discriminator for survival using the
discrimination index-score C. P-value is based on ranking among
100,000 permutations. q-value is calculated by Storey's method.

| | SEQ | | | |
|---|---|---|---|---|
| microRNA | ID NO: | C | p-value | q-value |
| hsa-miR-29c* | 19 | 0.726 | 5.00E−06 | 0.0024 |
| hsa-miR-130b | 9 | 0.317 | 0.002 | 0.0024 |
| hsa-miR-320a | 15 | 0.343 | 0.007 | 0.1163 |
| hsa-miR-140-3p | 11 | 0.639 | 0.011 | 0.3772 |
| hsa-miR-151-3p | 13 | 0.364 | 0.014 | 0.3772 |

As apparent from table 4, relatively high expression of either hsa-miR-29c* (SEQ ID NO: 19) and hsa-140-3p (SEQ ID NO: 11), with C-scores above 0.5, is predictive of longer survival. Relatively high expression of any hsa-miR-130b (SEQ ID NO: 9), hsa-miR-320a (SEQ ID NO: 15) and hsa-151-3p (SEQ ID NO: 13), with C-scores below 0.5, is predictive of shorter survival.

Finally, univariate Cox regression was used to determine the importance of each the expressed microRNAs as a prognosticator. Cox univariate analysis showed a reduced hazard for miR-29c* (SEQ ID NO: 19) with exp(β)=0.33, which was significant (p=7·10⁻⁴) and an increased hazard for miR-130b (SEQ ID NO: 9) (exp(β)=2.56; p=1·10⁻⁴).

### Example 3

### qRT-PCR Results

The microRNAs indicated in Table 5 were selected for quantitative real-time PCR (qRT-PCR) analysis. MicroRNA amounts were quantified using a qRT-PCR method as follows: RNA was incubated in the presence of poly (A) polymerase (PAP; Takara-2180A), MnCl₂, and ATP for 1 hour at 37° C. Then, using an oligodT primer harboring a consensus sequence, reverse transcription was performed on total RNA using SuperScript II RT (Invitrogen, Carlsbad, Calif.). Next, the cDNA was amplified by RT-PCR; this reaction contained a microRNA-specific forward primer, a TaqMan probe complementary to the 3′ of the specific microRNA sequence as well as to part of the polyA adaptor sequence, and a universal reverse primer complementary to the consensus 3′ sequence of the oligodT tail.

The cycle threshold (Ct, the PCR cycle at which probe signal reaches the threshold) was determined for each microRNA.

TABLE 5
Sequences used in RT-PCR

| | | SEQ | | SEQ |
|---|---|---|---|---|
| MiR | Forward primer | ID | MGB probes | ID |
| name | sequence | NO: | sequence | NO: |
| hsa- | CAGTCATTTGGCTAGC | 78 | CCGTTTTTTTTT | 101 |
| miR-29c | ACCATTTGAAAT | | TTTAACCGATT | |
| hsa- | CAGTCATTTGGCTTCA | 79 | CCGTTTTTTTTT | 102 |
| miR-26b | AGTAATTCAGGA | | TTTACCTATCC | |
| hsa-let- | CAGTCATTTGGGTGAG | 80 | CCGTTTTTTTTT | 103 |
| 7g | GTAGTAGTTTGT | | TTTACTGTACA | |
| hsa- | CAGTCATTTGGCTAAA | 81 | CCGTTTTTTTTT | 104 |
| miR- | GTGCTGACAGTG | | TTTATCTGCAC | |
| 106b | | | | |
| hsa- | CAGTCATTTGGCTACA | 82 | CCGTTTTTTTTT | 105 |
| miR-101 | GTACTGTGATAA | | TTTCAGTTATC | |
| hsa- | CAGTCATTTGGCTGTA | 83 | CCGTTTTTTTTT | 106 |
| miR-30e | AACATCCTTGAC | | TTTCTTCCAGT | |
| hsa- | CAGTCATTTGGCTAGC | 84 | TCCGTTTTTTTT | 107 |
| miR-29b | ACCATTTGAAAT | | TTTTAACACTGA | |
| hsa- | CAGTCATTTGGGTAAT | 85 | CCGTTTTTTTTT | 108 |
| miR- | ACTGCCTGGTAA | | TTTCATCATTA | |
| 200b | | | | |
| hsa- | CAGTCATTTGGGTAAC | 86 | CCGTTTTTTTTT | 109 |
| miR- | ACTGTCTGGTAA | | TTTAACATCGT | |
| 200a | | | | |
| hsa- | CAGTCATTTGGCTCAG | 87 | CCGTTTTTTTTT | 110 |
| miR- | TGCACTACAGAA | | TTTACAAAGTT | |
| 148a | | | | |
| hsa- | AGGGACGGGACGCGG | 88 | AAAACCGATAGT | 111 |
| miR- | TGCAGTG | | GAGTCG | |
| 92b* | | | | |
| hsa- | CAGTCATTTGGCCGCA | 89 | CCGTTTTTTTTT | 112 |
| miR- | TCCCCTAGGGCA | | TTTACACCAAT | |
| 324-5p | | | | |
| hsa- | CAGTCATTTGGGAACA | 90 | CCGTTTTTTTTT | 113 |
| miR- | TTCATTGCTGTC | | TTTACCCACCG | |
| 181b | | | | |
| hsa- | CAGTCATTTGGCCAGC | 91 | CCGTTTTTTTTT | 114 |
| miR-424 | AGCAATTCATGT | | TTTCAAAACAT | |
| hsa- | CAGTCATTTGGCCATT | 92 | CCGTTTTTTTTT | 115 |
| miR-25 | GCACTTGTCTCG | | TTTCAGACCGA | |
| hsa- | CAGTCATTTGGCTCTG | 93 | CCGTTTTTTTTT | 116 |
| miR-149 | GCTCCGTGTCTT | | TTTGGGAGTGA | |
| hsa- | CAGTCATTTGGCTATT | 94 | CGTTTTTTTTTT | 117 |
| miR-92a | GCACTTGTCCCG | | TTACAGGCCG | |
| hsa- | CAGTCATTTGGCAACT | 95 | CGTTTTTTTTTT | 118 |
| miR- | GGCCCTCAAAGT | | TTAGCGGGAC | |
| 193b | | | | |
| hsa- | CAGTCATTTGGGTATT | 96 | CGTTTTTTTTTT | 119 |
| miR-92b | GCACTCGTCCCG | | TTGGAGGCCG | |
| hsa- | CAGTCATTTGGCAGCT | 97 | CGTTTTTTTTTT | 120 |
| miR-138 | GGTGTTGTGAAT | | TTCGGCCTGA | |
| hsa- | CAGTCATTTGGCTTCA | 98 | CCGTTTTTTTTT | 121 |
| miR-27a | CAGTGGCTAAGT | | TTTGCGGAACT | |
| hsa- | CAGTCATTTGGCATCA | 99 | CCGTTTTTTTTT | 122 |
| miR-23a | CATTGCCAGGGA | | TTTGGAAATCC | |
| hsa- | CAGTCATTTGGCTAAG | 100 | CCGTTTTTTTTT | 123 |
| miR-18a | GTGCATCTAGTG | | TTTCTATCTGC | |

The microRNAs indicated in Table 6 were identified using log rank p-value of the Kaplan-Meier comparing the 1^{st} and 3^{rd} tertiles (of 81 fresh-frozen and FFPE samples) by expression level of the microRNA.

TABLE 6

| | MiR | Hairpin | p-value | Up (+) or down (−) |
|---|---|---|---|---|
| | SEQ ID | SEQ | (Kaplan-meier | regulated in good |
| MiR Name | NO: | ID NO: | logrank) | prognosis |
| hsa-miR-29c | 29 | 20 | 0.0028 | + |
| hsa-miR-26b | 30 | 52 | 0.003 | + |
| hsa-let-7g | 31 | 53 | 0.0066 | + |
| hsa-miR- | 32 | 54 | 0.0075 | + |
| 106b | | | | |
| hsa-miR-101 | 33 | 55, 56 | 0.0048 | + |
| hsa-miR-30e | 34 | 57 | 0.001 | + |
| hsa-miR-29b | 35 | 58, 59 | 0.0001 | + |
| hsa-miR- | 36 | 60 | 0.012 | − |
| 92b* | | | | |
| hsa-miR- | 37 | 61 | 0.0195 | − |
| 324-5p | | | | |
| hsa-miR- | 38 | 62, 63 | 0.0437 | − |
| 181b | | | | |
| hsa-miR-424 | 39 | 64 | 0.0494 | − |
| hsa-miR-25 | 40 | 65 | 0.0035 | − |
| hsa-miR-149 | 41 | 66 | 0.0082 | − |
| hsa-miR-92a | 42 | 67, 68 | 0.0194 | − |
| hsa-miR- | 43 | 69 | 0.0026 | − |
| 193b | | | | |
| hsa-miR-92b | 44 | 60 | 0.0016 | − |

The microRNAs indicated in Table 7 were found significantly differentially expressed (pValue<0.05) by 2-sided t-test comparing expression levels of 8 long survivors (more than 18 month to death) and 11 quick progressors (less than 12 month to death). All tested samples were fresh-frozen samples of stage 2 patients.

TABLE 7

| | | Normalized median | | | | Up (+) or |
|---|---|---|---|---|---|---|
| | MiR | expression (50-CT) | | | | down (−) |
| | SEQ | Good | Bad | | | regulated |
| MiR | ID | prognosis | prognosis | Fold | p-value | in good |
| Name | NO: | group | group | Change | (t-test) | prognosis |
| hsa-miR- | 45 | 15.715 | 12.945 | 6.82 | 0.0017 | + |
| 200b | | | | | | |
| hsa-miR- | 46 | 15.5 | 13.126 | 5.21 | 0.0016 | + |
| 200a | | | | | | |
| hsa-miR- | 47 | 22.129 | 20.736 | 2.63 | 0.0077 | + |
| 148a | | | | | | |
| hsa-miR- | 48 | 13.46 | 15.539 | 4.22 | 0.0019 | − |
| 138 | | | | | | |
| hsa-miR- | 49 | 24.46 | 25.54 | 2.12 | 0.0013 | − |
| 27a | | | | | | |
| hsa-miR- | 50 | 24.98 | 25.874 | 1.86 | 0.0092 | − |
| 23a | | | | | | |
| hsa-miR- | 51 | 17.124 | 18.012 | 1.85 | 0.01 | − |
| 18a | | | | | | |

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It should be understood that the detailed description and specific examples, while indicating preferred embodiments of the invention, are given by way of illustration only, since various changes and modifications within the spirit and scope of the invention will become apparent to those skilled in the art from this detailed description.

1. A method for determining a prognosis for mesothelioma in a subject, the method comprising:
(a) providing a biological sample from the subject;
(b) determining the expression level in said sample of a nucleic acid sequence selected from the group consisting of SEQ ID NOS: 1-16, 21-77 and sequences at least about 80% identical thereto; and
(c) comparing said expression level to a threshold expression level, wherein the comparison of the expression level of said nucleic acids to said threshold expression level is indicative of the prognosis of said subject.

2. The method of claim 1, wherein the nucleic acid sequence is selected from the group consisting of SEQ ID NOS: 1-4, 11-12, 21, 22, 27-35, 45-47, 52-59, 70-72 and sequences at least about 80% identical thereto, and wherein an increased expression level of any of said nucleic acid sequence compared to said threshold expression level is indicative of good prognosis of said subject.

3. The method of claim 1, wherein the nucleic acid sequence is selected from the group consisting of SEQ ID NOS: 5-10, 13-16, 23-26, 36-44, 48-51, 60-69, 73-77 and sequences at least about 80% identical thereto and wherein an increased expression level of any of said nucleic acid sequence compared to said threshold expression level is indicative of poor prognosis of said subject.

4. The method of claim 1, wherein the subject is a human.

5. The method of claim 1, wherein said method is used to determine a course of treatment for said subject.

6. The method of claim 1, wherein said biological sample is selected from the group consisting of bodily fluid, a cell line and a tissue sample.

7. The method of claim 6, wherein said bodily fluid is blood.

8. The method of claim 6, wherein said tissue is a fresh, frozen, fixed, wax-embedded or formalin fixed paraffin-embedded (FFPE) tissue.

9. The method of claim 8, wherein said tissue is mesothelium.

10. The method of claim 1, wherein the expression levels are determined by a method selected from the group consisting of nucleic acid hybridization, nucleic acid amplification, and a combination thereof.

11. The method of claim 10, wherein the nucleic acid hybridization is performed using a solid-phase nucleic acid biochip array or in situ hybridization.

12. The method of claim 10, wherein the nucleic acid amplification method is real-time PCR.

13. The method of claim 12, wherein the real-time PCR method comprises forward and reverse primers.

14. The method of claim 13, wherein said forward primer comprises a nucleic acid sequence that is partially complementary to a sequence selected from SEQ ID NO: 116, 21-77 to a fragment thereof or to a sequence at least about 80% identical thereto.

15. The method of claim 13, wherein said forward primer comprises a sequence selected from the group consisting of SEQ ID NOS: 78-100 and sequences at least about 80% identical thereto.

16. The method of claim 13, wherein said reverse primer comprises SEQ ID NO: 124 and sequences at least about 80% identical thereto.

17. The method of claim 13, wherein the real-time PCR method further comprises a probe.

18. The method of claim 17, wherein the probe comprises a sequence complementary to SEQ ID NOS: 1-16, 21-77, to a fragment thereof or to a sequence at least about 80% identical thereto.

19. The method of claim 17, wherein the probe comprises a sequence selected from the group consisting of SEQ ID NOS: 101-123 and sequences at least about 80% identical thereto.

20. A kit for determining a prognosis of a subject with mesothelioma, said kit comprising forward and reverse primers and a probe.

21. The kit of claim 20, wherein said probe comprises a nucleic acid sequence that is complementary to a sequence selected from SEQ ID NO: 1-16, 21-77, to a fragment thereof or to a sequence at least about 80% identical thereto.

22. The kit of claim 20, wherein said probe comprises a sequence selected from the group consisting of SEQ ID NOS: 101-123 and sequences at least about 80% identical thereto.

23. The kit of claim 20, wherein said forward primer is partially complementary to SEQ ID NOS: 1-16, 21-77 to a fragment thereof or to a sequence at least about 80% identical thereto.

24. The kit of claim 20, wherein said forward primer comprises a sequence selected from the group consisting of SEQ ID NOS: 78-100 and sequences at least about 80% identical thereto.

25. The kit of claim 20, wherein said reverse primer comprises a sequence selected from the group consisting of SEQ ID NOS: 124 and sequences at least about 80% identical thereto

26. The kit of claim 17, wherein the kit comprises reagents for performing in situ hybridization analysis.
